(19) United States
(12) Patent Application Publication
(10) Pub. No.: US 2024/0226916 A9
(43) Pub. Date: Jul. 11, 2024

(54) Systems And Methods For Collecting A Platelet Product Having A Target Concentration

(71) Applicant: Fenwal Inc., Lake Zurich, IL (US)

(72) Inventors: Benjamin E. Kusters, Pleasant Prairie, WI (US); Kyungyoon Min, Kildeer, IL (US)

(21) Appl. No.: 18/378,679

(22) Filed: Oct. 11, 2023

Related U.S. Application Data
(60) Provisional application No. 63/380,607, filed on Oct. 24, 2022.

(51) Int. Cl. B04B 11/02; A61M 1/36; B01D 36/04; B01D 63/16; B04B 5/04

(52) U.S. Cl. CPC B04B 11/02; A61M 1/3696; B01D 36/045; B01D 63/16; B04B 5/0442; A61M 2202/0415; A61M 2202/0427; A61M 2202/0429; A61M 2205/3303

(57) ABSTRACT

Systems and methods are provided for collecting a platelet product having a target concentration. First and second target concentrations are first selected. Blood is then separated into red blood cells and platelet-rich plasma, with the platelet-rich plasma then being separated into platelet-poor plasma and platelet concentrate. At least a portion of the platelet concentrate is collected in a container as a platelet product having an actual platelet concentration, attempting to collect platelet concentrate having the first target concentration. After separation of the blood has been completed, at least a portion of the platelet-poor plasma and/or an additive solution is pumped into the container to decrease the concentration of the platelet product from the actual platelet concentration to the second target concentration.

### CROSS-REFERENCE TO RELATED APPLICATIONS

This application claims the benefit of and priority of U.S. Provisional Patent Application Ser. No. 63/380,607, filed Oct. 24, 2022, the contents of which are incorporated by reference herein.

### BACKGROUND

### Field of the Disclosure

The present subject matter relates to systems and methods for separating blood so as to produce a platelet product. More particularly, the present subject matter relates to collection of a platelet product having a target platelet concentration.

### Description of Related Art

Various blood processing systems now make it possible to collect particular blood constituents, instead of whole blood, from a blood source. Typically, in such systems, whole blood is drawn from a blood source, the particular blood component or constituent is separated, removed, and collected, and the remaining blood constituents are returned to the blood source. Removing only particular constituents is advantageous when the blood source is a human donor, because potentially less time is needed for the donor's body to return to pre-donation levels, and donations can be made at more frequent intervals than when whole blood is collected. This increases the overall supply of blood constituents, such as plasma and platelets, made available for transfer and/or therapeutic treatment.

According to one approach, whole blood may be separated into its constituents through centrifugation. This requires that the whole blood be passed through a centrifuge after it is withdrawn from, and before it is returned to, the blood source. To reduce contamination and possible infection (if the blood source is a human donor or patient), the blood is preferably processed within a sealed, sterile fluid flow circuit during the centrifugation process. Typical disposable flow circuits include a separation chamber portion, which is mounted in cooperation on a durable, reusable assembly containing the hardware (centrifuge, drive system, pumps, valve actuators, programmable controller, and the like) that rotates the separation chamber and controls the flow through the fluid circuit.

The centrifuge rotates the separation chamber of the disposable flow circuit during processing, causing the heavier (greater specific gravity) components of the whole blood in the separation chamber, such as red blood cells, to move radially outwardly away from the center of rotation toward the outer or “high-G” wall of the separation chamber. The lighter (lower specific gravity) components, such as plasma, migrate toward the inner or “low-G” wall of the separation chamber. The boundary that forms between the heavier and lighter components in the separation chamber is commonly referred to as the interface. Various ones of these components can be selectively removed from the whole blood by providing appropriately located channeling structures and outlet ports in the flow circuit. For example, in one blood separation procedure, platelet-rich plasma may be separated from the other blood components and collected, with the non-targeted blood components and a replacement fluid being returned to the blood source.

There is another class of devices, based on the use of a membrane, that has been used for plasmapheresis (i.e., separating plasma from whole blood). More specifically, this type of device employs relatively rotating surfaces, at least one or which carries a porous membrane. Typically, the device employs an outer stationary housing and an internal spinning rotor covered by a porous membrane.

Well-known plasmapheresis devices include the AUTOPHERESIS-C® and AURORO® separators sold by Fenwal, Inc. of Lake Zurich, Illinois, which is an affiliate of Fresenius Kabi AG of Bad Homburg, Germany. A detailed description of an exemplary spinning membrane separator may be found in U.S. Pat. No. 5,194,145, which is incorporated by reference herein. This patent describes a membrane-covered spinner having an interior collection system disposed within a stationary shell. Blood is fed into an annular space or gap between the spinner and the shell. The blood moves along the longitudinal axis of the shell toward an exit region, with plasma passing through the membrane and out of the shell into a collection bag. The remaining blood components, primarily red blood cells, platelets, and white blood cells, move to the exit region between the spinner and the shell and then are typically returned to the donor.

More recently, integrated systems incorporating both centrifugal separation and spinning membrane separation have been developed. Such a system is described in detail in PCT Patent Application Publication No. WO 2018/053217, which is incorporated herein by reference.

According to one conventional approach to platelet collection, a blood separation system will collect all platelets that have been separated from blood (which may include the platelets being separated from a previously separated blood component, such as platelet-rich plasma), regardless of the platelet concentration (which may be referred to herein simply as “concentration”) of the platelet concentrate. Subsequently, fluid is added to the platelet concentrate to produce a platelet product having a target volume and concentration. This fluid is typically platelet-poor plasma or a platelet additive solution (commonly referred to as “PAS”).

One possible disadvantage of this conventional approach (which targets a desired final platelet concentration throughout steady state separation) is that it is vulnerable to pump-driven flow inaccuracies. For example, if a target final platelet concentration is 1500e3 platelets/μL and the pump system actually produces a product having a different concentration, the concentration of the platelet concentrate produced by the blood separation will have an improper concentration that cannot be corrected by the system. Thus, it would be advantageous to provide a platelet collection procedure that is unaffected by (or at least less vulnerable to) pump inaccuracies.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately as set forth in the claims appended hereto.

In one aspect, a blood separation device includes a pump system, a valve system, a separation system, and a controller that is configured and/or programmed to control the operation of the pump system, the valve system, and the separation system to execute a platelet collection procedure. The procedure includes selecting a first target concentration and a second target concentration, then separating blood into red blood cells and platelet-rich plasma and separating the platelet-rich plasma into platelet-poor plasma and platelet concentrate. At least a portion of the platelet concentrate is collected in a container as a platelet product having an actual platelet concentration, with the pump system being controlled so as to attempt to collect platelet concentrate having the first target concentration. After the separation of blood has been completed, at least a portion of the platelet-poor plasma and/or an additive solution is pumped into the container to decrease the concentration of the platelet product from the actual platelet concentration to the second target concentration.

In another aspect, a blood separation device includes a pump system, a valve system, a centrifugal separator, a spinning membrane separator drive unit, and a controller that is configured and/or programmed to control the operation of the pump system, the valve system, the centrifugal separator, and the spinning membrane separator drive unit to execute a platelet collection procedure. The procedure includes selecting a first target concentration and a second target concentration, pumping blood from a blood source into the centrifugal separator, and then separating the blood in the centrifugal separator into red blood cells and platelet-rich plasma. At least a portion of the platelet-rich plasma is pumped from the centrifugal separator into the spinning membrane separator drive unit, where it is separated into platelet-poor plasma and platelet concentrate, with at least a portion of the platelet-poor plasma being collected in a first container as collected plasma and at least a portion of the platelet concentrate being collected in a second container as a platelet product having an actual platelet concentration. The pump system is controlled so as to attempt to collect platelet concentrate having the first target concentration. After the separation of blood has been completed, at least a portion of the collected plasma and/or an additive solution is pumped into the second container to decrease the concentration of the platelet product from the actual platelet concentration to the second target concentration.

In yet another aspect, a blood separation method includes selecting a first target concentration and a second target concentration. Blood is separated into red blood cells and platelet-rich plasma, with the platelet-rich plasma being separated into platelet-poor plasma and platelet concentrate, and collecting at least a portion of the platelet concentrate in a container as a platelet product having an actual platelet concentration. It is attempted to collect platelet concentrate having the first target concentration. After the separation of blood has been completed, at least a portion of the platelet-poor plasma and/or an additive solution is pumped into the container to decrease the concentration of the platelet product from the actual platelet concentration to the second target concentration.

### BRIEF DESCRIPTION OF THE DRAWINGS

FIG. 1 is a perspective view of an exemplary blood separation device that comprises a component of a blood separation system according to an aspect of the present disclosure;

FIG. 2 is a schematic view of an exemplary disposable fluid flow circuit that may be mounted to the blood separation device of FIG. 1 to complete a blood separation system according to an aspect of the present disclosure; and

FIGS. 3-7 are schematic views of the fluid flow circuit of FIG. 2 mounted on the blood separation device of FIG. 1, showing the system carrying out different fluid flow tasks in connection with separation of platelets from blood and collection of a platelet product having a target concentration.

### DESCRIPTION OF THE ILLUSTRATED EMBODIMENTS

The embodiments disclosed herein are for the purpose of providing an exemplary description of the present subject matter. They are, however, only exemplary, and the present subject matter may be embodied in various forms. Therefore, specific details disclosed herein are not to be interpreted as limiting the subject matter as defined in the accompanying claims.

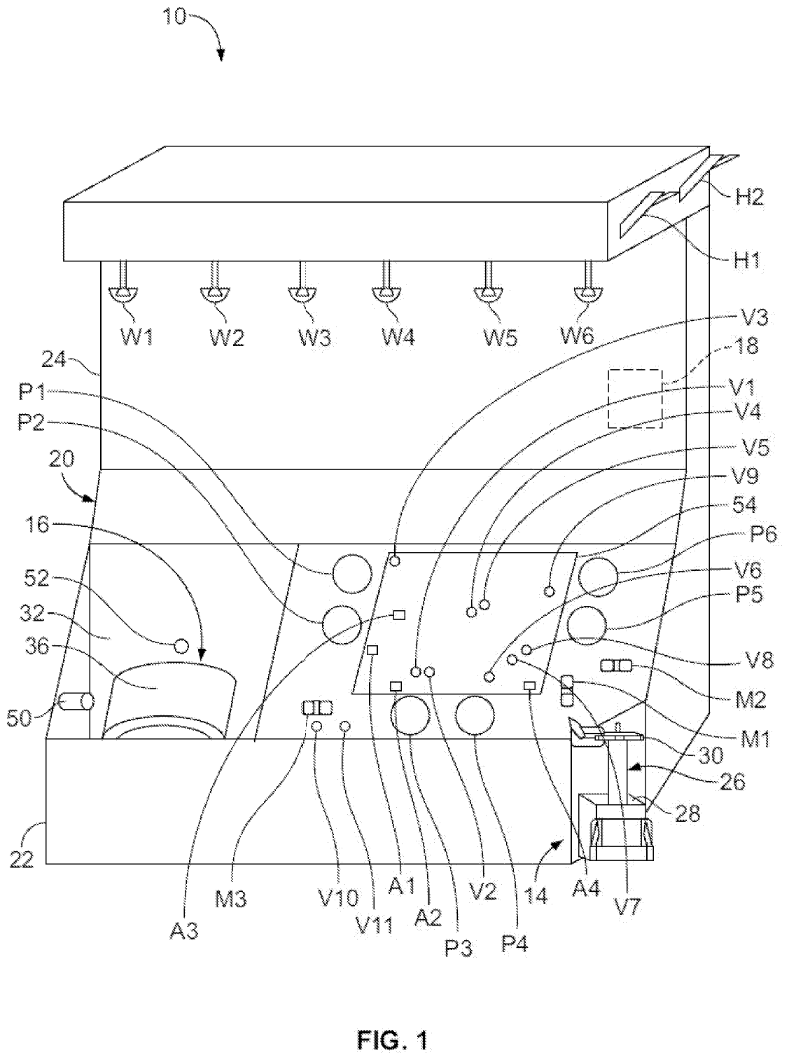

FIGS. 1-9 show components of a blood or fluid separation system that embodies various aspects of the present subject matter. While the system may be referred to herein as a “blood processing system” or a “blood separation system” and examples will be given of various ways in which the system may be used to separate blood into its component parts, it should be understood that systems according to the present disclosure can be used for processing a variety of fluids, which may include bodily fluids and non-bodily fluids.

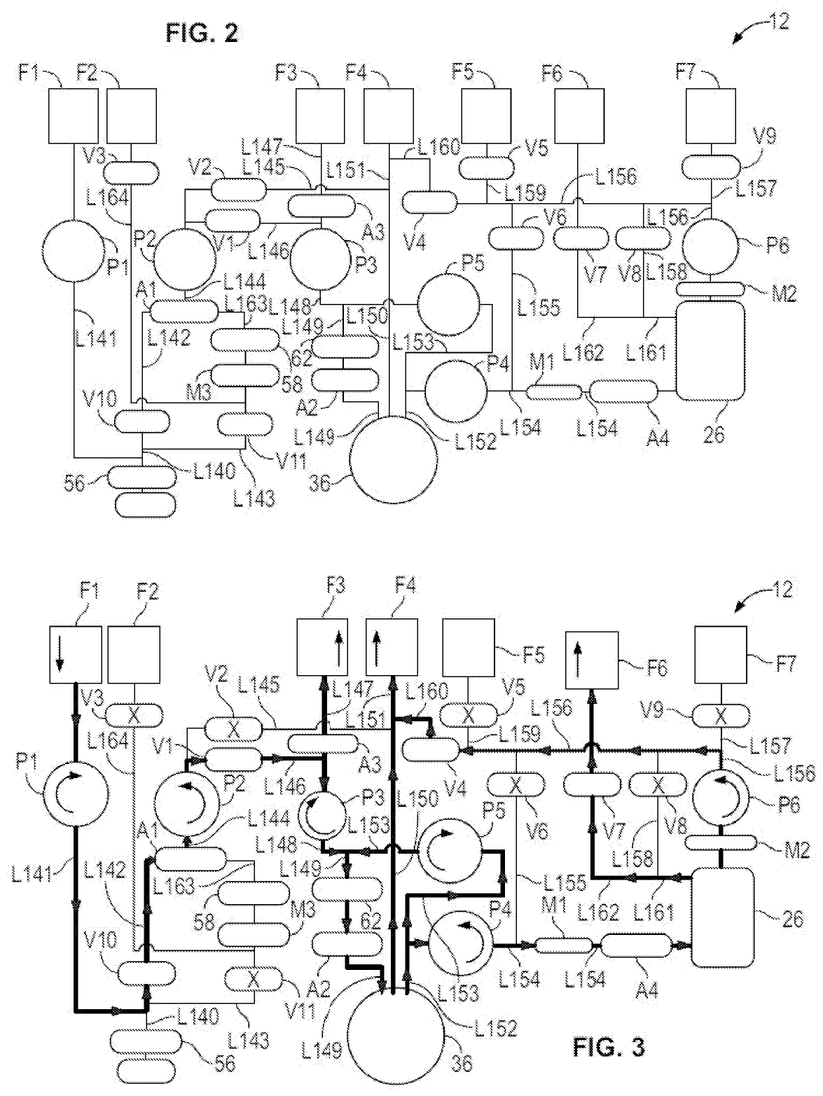

Generally speaking, the system includes two principal components, a durable and reusable blood separation device 10 (FIG. 1) and a disposable fluid flow circuit 12 (FIG. 2). The blood separation device 10 includes a spinning membrane separator drive unit 14, a centrifuge or centrifugal separator 16, additional components that control fluid flow through the disposable flow circuit 12, and a controller 18, which governs the operation of the other components of the blood separation device 10 to perform a blood processing and collection procedure, as will be described in greater detail. While the principles described herein may be employed when using the blood separation device 10 of FIG. 1, it should be understood that these same principles may be applied to other blood separation devices, including devices employing single separation technologies or approaches.

### I. The Durable Blood Separation Device

The blood separation device 10 (FIG. 1) is configured as a durable item that is capable of long-term use. It should be understood that the blood separation device 10 of FIG. 1 is merely exemplary of one possible configuration and that blood separation devices according to the present disclosure may be differently configured.

In the illustrated embodiment, the blood separation device 10 is embodied in a single housing or case 20. The illustrated case 20 includes a generally horizontal portion 22 (which may include an inclined or angled face or upper surface for enhanced visibility and ergonomics) and a generally vertical portion 24. The spinning membrane separator drive unit 14 and the centrifugal separator 16 are shown as being incorporated into the generally horizontal portion 22 of the case 20, while the controller 18 is shown as being incorporated into the generally vertical portion 24.

### A. Spinning Membrane Separator Drive Unit

The blood separation device 10 includes a spinner support or spinning membrane separator drive unit 14 for accommodating a generally cylindrical spinning membrane separator 26 of the fluid flow circuit 12. U.S. Pat. No. 5,194,145 describes an exemplary spinning membrane separator drive unit that would be suitable for incorporation into the blood separation device 10, but it should be understood that the spinning membrane separator drive unit 14 may be differently configured without departing from the scope of the present disclosure.

The illustrated spinning membrane separator drive unit 14 has a base 28 configured to receive a lower portion of the spinning membrane separator 26 and an upper end cap 30 to receive an upper portion of the spinning membrane separator 26. Preferably, the upper end cap 30 is positioned directly above the base 28 to orient a spinning membrane separator 26 received by the spinning membrane separator drive unit 14 vertically and to define a vertical axis about which the spinning membrane separator 26 is spun. While it may be advantageous for the spinning membrane separator drive unit 14 to vertically orient a spinning membrane separator 26, it is also within the scope of the present disclosure for the spinning membrane separator 26 to be differently oriented when mounted to the blood separation device 10.

In one embodiment, one of the base 28 and upper end cap 30 of the spinning membrane separator drive unit 14 is movable with respect to the other, which may allow differently sized spinning membrane separators 26 to be received by the spinning membrane separator drive unit 14. For example, the upper end cap 30 may be translated vertically with respect to the base 28 and locked in a plurality of different positions, with each locking position corresponding to a differently sized spinning membrane separator 26.

At least one of the base 28 and the upper end cap 30 is configured to spin one or more components of the spinning membrane separator 26 about the axis defined by the spinning membrane separator drive unit 14. The mechanism by which the spinning membrane separator drive unit 14 spins one or more components of the spinning membrane separator 26 may vary without departing from the scope of the present disclosure. In one embodiment, a component of the spinning membrane separator 26 to be spun includes at least one element configured to be acted upon by a magnet (e.g., a metallic material), while the spinning membrane separator drive unit 14 includes a magnet (e.g., a series of magnetic coils or semi-circular arcs). By modulating the magnetic field acting upon the aforementioned element of the spinning membrane separator 26, the component or components of the spinning membrane separator 26 may be made to spin in different directions and at varying speeds. In other embodiments, different mechanisms may be employed to spin the component or components of the spinning membrane separator 26.

Regardless of the mechanism by which the spinning membrane separator drive unit 14 spins the component or components of the spinning membrane separator 26, the component or components of the spinning membrane separator 26 is/are preferably spun at a speed that is sufficient to create Taylor vortices in a gap between the spinning component and a stationary component of the spinning membrane separator 26 (or a component that spins at a different speed). Fluid to be separated within the spinning membrane separator 26 flows through this gap, and filtration may be dramatically improved by the creation of Taylor vortices.

### B. Centrifugal Separator

As for the centrifugal separator 16, it includes a centrifuge compartment 32 that receives a centrifugal separation chamber 36 of the fluid flow circuit 12, as well as other components of the centrifugal separator 16. Further details as to the centrifugal separator are set forth in WO 2018/053217, referenced and incorporated herein by reference above.

Blood is introduced into the centrifugal separation chamber 36 by an umbilicus, with the blood being separated (e.g., into a layer of less dense components, such as platelet-rich plasma, and a layer of more dense components, such as packed red blood cells and white blood cells) within the centrifugal separation chamber 36 as a result of centrifugal forces as it rotates. Components of an interface monitoring system may be positioned within the centrifuge compartment 32 to oversee separation of blood within the centrifugal separation chamber 36. The interface monitoring system may include a light source 50 and a light detector 52, which is positioned and oriented to receive at least a portion of the light emitted by the light source 50.

The orientation of the various components of the interface monitoring system depends at least in part on the particular configuration of the centrifugal separation chamber 36. In general, though, the light source 50 emits a light beam (e.g., a laser light beam) through the separated blood components within the centrifugal separation chamber 36 (which may be formed of a material that substantially transmits the light or at least a particular wavelength of the light without absorbing it). A portion of the light reaches the light detector 52, which transmits a signal to the controller 18 that is indicative of the location of an interface between the separated blood components. If the controller 18 determines that the interface is in the wrong location (which can affect the separation efficiency of the centrifugal separator 16 and/or the quality of the separated blood components), then it can issue commands to the appropriate components of the blood separation device 10 to modify their operation so as to move the interface to the proper location.

### C. Other Components Of The Blood Separation Device

In addition to the spinning membrane separator drive unit 14 and the centrifugal separator 16, the blood separation device 10 may include other components compactly arranged to aid blood processing.

The generally horizontal portion 22 of the case 20 of the illustrated blood separation device 10 includes a cassette station 54, which accommodates a flow control cassette of the fluid flow circuit 12. In one embodiment, the cassette station 54 is similarly configured to the cassette station of U.S. Pat. No. 5,868,696 (which is incorporated herein by reference), but is adapted to include additional components and functionality. The illustrated cassette station 54 includes a plurality of clamps or valves V1-V9, which move between a plurality of positions (e.g., between a retracted or lowered position and an actuated or raised position) to selectively contact or otherwise interact with corresponding valve stations of the flow control cassette of the fluid flow circuit 12. Depending on the configuration of the fluid flow circuit 12, its cassette may not include a valve station for each valve V1-V9 of the cassette station 54, in which case fewer than all of the valves V1-V9 will be used in a separation procedure.

In the actuated position, a valve V1-V9 engages the associated valve station to prevent fluid flow through that valve station (e.g., by closing one or more ports associated with the valve station, thereby preventing fluid flow through that port or ports). In the retracted position, a valve V1-V9 is disengaged from the associated valve station (or less forcefully contacts the associated valve station than when in the actuated position) to allow fluid flow through that valve station (e.g., by opening one or more ports associated with the valve station, thereby allowing fluid flow through that port or ports). Additional clamps or valves V10 and V11 may be positioned outside of the cassette station 54 to interact with portions of valve stations (which may be lengths of tubing) of the fluid flow circuit 12 to selectively allow and prevent fluid flow therethrough. The valves V1-V9 and corresponding valve stations of the cassette station 54 and cassette may be differently configured and operate differently from the valves V10 and V11 and the valve stations that are spaced away from the cassette station 54.

The cassette station 54 may be provided with additional components, such as pressure sensors A1-A4, which interact with sensor stations of the cassette to monitor the pressure at various locations of the fluid flow circuit 12. For example, if the blood source is a human donor, one or more of the pressure sensors A1-A4 may be configured to monitor the pressure of the donor's vein during blood draw and return. Other pressure sensors A1-A4 may monitor the pressure of the spinning membrane separator 26 and the centrifugal separation chamber 36. The controller 18 may receive signals from the pressure sensors A1-A4 that are indicative of the pressure within the fluid flow circuit 12 and, if a signal indicates a low- or high-pressure condition, the controller 18 may initiate an alarm or error condition to alert an operator to the condition and/or to attempt to bring the pressure to an acceptable level without operator intervention.

The blood separation device 10 may also include a plurality of pumps P1-P6 to cause fluid to flow through the fluid flow circuit 12. The pumps P1-P6 may be differently or similarly configured and/or function similarly or differently from each other. In the illustrated embodiment, the pumps P1-P6 are configured as peristaltic pumps, which may be generally configured as described in U.S. Pat. No. 5,868,696. Each pump P1-P6 engages a different tubing loop extending from a side surface of the flow control cassette and may be selectively operated under command of the controller 18 to cause fluid to flow through a portion of the fluid flow circuit 12, as will be described in greater detail below. In one embodiment, all or a portion of the cassette station 54 may be capable of translational motion in and out of the case 20 to allow for automatic loading of the tubing loops into the associated pump P1-P6.

The illustrated blood separation device 10 also includes a spinner inlet sensor M1 for determining one or more properties of a fluid flowing into a spinning membrane separator 26 mounted within the spinning membrane separator drive unit 14. If the fluid flowing into the spinning membrane separator 26 is whole blood (which may include anticoagulated whole blood), the spinner inlet sensor M1 may be configured to determine the hematocrit of the blood flowing into the spinning membrane separator 26. If the fluid flowing into the spinning membrane separator 26 is platelet-rich plasma, the spinner inlet sensor M1 may be configured to determine the platelet concentration of platelet-rich plasma flowing into the spinning membrane separator 26. The spinner inlet sensor M1 may detect the one or more properties of a fluid by optically monitoring the fluid as it flows through tubing of the fluid flow circuit 12, or by any other suitable approach. The controller 18 may receive signals from the spinner inlet sensor M1 that are indicative of the one or more properties of fluid flowing into the spinning membrane separator 26 and use the signals to optimize the separation procedure based upon that property or properties. If the property or properties is/are outside of an acceptable range, then the controller 18 may initiate an alarm or error condition to alert an operator to the condition. A suitable device and method for monitoring hematocrit and/or platelet concentration is described in U.S. Pat. No. 6,419,822 (which is incorporated herein by reference), but it should be understood that a different approach may also be employed for monitoring hematocrit and/or platelet concentration of fluid flowing into the spinning membrane separator 26.

The illustrated blood separation device 10 further includes a spinner outlet sensor M2, which accommodates tubing of the fluid flow circuit 12 that flows a separated blood component out of the spinning membrane separator 26. The spinner outlet sensor M2 monitors the fluid to determine one or more properties of the fluid, and may do so by optically monitoring the fluid as it flows through the tubing or by any other suitable approach. In one embodiment, separated plasma flows through the tubing, in which case the spinner outlet sensor M2 may be configured to determine the amount of cellular blood components in the plasma and/or whether the plasma is hemolytic and/or lipemic. This may be done using an optical monitor of the type described in U.S. Pat. No. 8,556,793 (which is incorporated herein by reference) that measures the optical density of the fluid in the associated tubing, or by any other suitable device and/or method.

The illustrated blood separation device also includes an air detector M3 (e.g., an ultrasonic bubble detector), which accommodates tubing of the fluid flow circuit 12 that flows fluid to a recipient. It may be advantageous to prevent air from reaching the recipient, whether a human recipient (e.g., the same human that serves as the blood source) or a non-human recipient (e.g., a storage bag or container), so the air detector M3 may transmit signals to the controller 18 that are indicative of the presence or absence of air in the tubing. If the signal is indicative of air being present in the tubing, the controller 18 may initiate an alarm or error condition to alert an operator to the condition and/or to take corrective action to prevent the air from reaching the recipient (e.g., by reversing the flow of fluid through the tubing or diverting flow to a vent location).

The generally vertical portion 24 of the case 20 may include a plurality of weight scales W1-W6 (six are shown, but more or fewer may be provided), each of which may support one or more fluid containers F1-F7 of the fluid flow circuit (FIGS. 2-7). The containers F1-F7 receive blood components separated during processing or intravenous fluids or additive fluids. Each weight scale W1-W6 transmits to the controller 18 a signal that is indicative of the weight of the fluid within the associated container F1-F7 to track the change of weight during the course of a procedure. This allows the controller 18 to process the incremental weight changes to derive fluid processing volumes and flow rates and subsequently generate signals to control processing events based, at least in part, upon the derived processing volumes. For example, the controller 18 may diagnose leaks and obstructions in the fluid flow circuit 12 and alert an operator.

The illustrated case 20 is also provided with a plurality of hooks or supports H1 and H2 that may support various components of the fluid flow circuit 12 or other suitably sized and configured objects.

### D. Controller

According to an aspect of the present disclosure, the blood separation device includes a controller 18, which is suitably configured and/or programmed to control operation of the blood separation device 10. In one embodiment, the controller 18 comprises a main processing unit (MPU), which can comprise, e.g., a Pentium™ type microprocessor made by Intel Corporation, although other types of conventional microprocessors can be used. In one embodiment, the controller 18 may be mounted inside the generally vertical portion 24 of the case 20, adjacent to or incorporated into an operator interface station (e.g., a touchscreen). In other embodiments, the controller 18 and operator interface station may be associated with the generally horizontal portion 22 or may be incorporated into a separate device that is connected (either physically, by a cable or the like, or wirelessly) to the blood separation device 10.

The controller 18 is configured and/or programmed to execute at least one blood processing application but, more advantageously, is configured and/or programmed to execute a variety of different blood processing applications. For example, the controller 18 may be configured and/or programmed to carry out one or more of the following: a double unit red blood cell collection procedure, a plasma collection procedure, a plasma/red blood cell collection procedure, a red blood cell/platelet/plasma collection procedure, a platelet collection procedure, and a platelet/plasma collection procedure. In the context of the present disclosure, the controller is programmed to carry out platelet collection procedures in which the resulting platelet product has a target concentration, as will be described in greater detail below.

More particularly, in carrying out these blood processing applications, the controller 18 is configured and/or programmed to control one or more of the following tasks: drawing blood into a fluid flow circuit 12 mounted to the blood separation device 10, conveying blood through the fluid flow circuit 12 to a location for separation (i.e., into a spinning membrane separator 26 or centrifugal separation chamber 36 of the fluid flow circuit 12), separating the blood into two or more components as desired, and conveying the separated components into storage containers, to a second location for further separation (e.g., into whichever of the spinning membrane separator 26 and centrifugal separation chamber 36 that was not used in the initial separation stage), or to a recipient (which may be a donor from which the blood was originally drawn).

This may include instructing the spinning membrane separator drive unit 14 and/or the centrifugal separator 16 to operate at a particular rotational speed and instructing a pump P1-P6 to convey fluid through a portion of the fluid flow circuit 12 at a particular flow rate. Hence, while it may be described herein that a particular component of the blood separation device 10 (e.g., the spinning membrane separator drive unit 14 or the centrifugal separator 16) performs a particular function, it should be understood that that component is being controlled by the controller 18 to perform that function.

As will be seen, the procedure described herein calls for the use of both the centrifugal separator 16 and the spinning membrane separator drive unit 14, in which case a properly programmed controller 18 is especially important to coordinate the operation of these two components, along with the other components of the blood separation device 10 to ensure that flow to and from the centrifugal separation chamber 36 and spinning membrane separator 26 is at the proper level and that the components are functioning properly to process the blood circulating through the fluid flow circuit 12.

Before, during, and after a procedure, the controller 18 may receive signals from various components of the blood separation device 10 (e.g., the pressure sensors A1-A4) to monitor various aspects of the operation of the blood separation device 10 and characteristics of the blood and separated blood components as they flow through the fluid flow circuit 12. If the operation of any of the components and/or one or more characteristics of the blood or separated blood components is outside of an acceptable range, then the controller 18 may initiate an alarm or error condition to alert the operator and/or take action to attempt to correct the condition. The appropriate corrective action will depend upon the particular error condition and may include action that is carried out with or without the involvement of an operator.

For example, the controller 18 may include an interface control module, which receives signals from the light detector 52 of the interface monitoring system. The signals that the controller 18 receives from the light detector 52 are indicative of the location of an interface between the separated blood components within the centrifugal separation chamber 36. If the controller 18 determines that the interface is in the wrong location, then it can issue commands to the appropriate components of the blood separation device 10 to modify their operation so as to move the interface to the proper location. For example, the controller 18 may instruct one of the pumps P1-P6 to cause blood to flow into the centrifugal separation chamber 36 at a different rate and/or for a separated blood component to be removed from the centrifugal separation chamber 36 at a different rate and/or for the centrifugal separation chamber 36 to be spun at a different speed by the centrifugal separator 16. Such control typically occurs regardless of whether the blood originates from a container or directly from a donor, and regardless of whether the components are directed into storage containers or returned to a donor or another living recipient.

If provided, an operator interface station associated with the controller 18 allows the operator to view on a screen or display (in alpha-numeric format and/or as graphical images) information regarding the operation of the system. The operator interface station also allows the operator to select applications to be executed by the controller 18, as well as to change certain functions and performance criteria of the system. If configured as a touchscreen, the screen of the operator interface station can receive input from an operator via touch-activation. Otherwise, if the screen is not a touchscreen, then the operator interface station may receive input from an operator via a separate input device, such as a computer mouse or keyboard. It is also within the scope of the present disclosure for the operator interface station to receive input from both a touchscreen and a separate input device, such as a keypad.

### II. The Disposable Fluid Flow Circuit

As for the fluid flow circuit or flow set 12 (FIGS. 2-7), it is intended to be a sterile, single use, disposable item. Before beginning a given blood processing and collection procedure, the operator loads various components of the fluid flow circuit 12 in the case 20 in association with the blood separation device 10. The controller 18 implements the procedure based upon preset protocols, taking into account other input from the operator. Upon completing the procedure, the operator removes the fluid flow circuit 12 from association with the blood separation device 10. The portions of the fluid flow circuit 12 holding the collected blood component or components (e.g., collection containers or bags) are removed from the case 20 and retained for storage, transfusion, or further processing. The remainder of the fluid flow circuit 12 is removed from the case 20 and discarded.

In the illustrated embodiment, the fluid flow circuit 12 includes a cassette, to which the other components of the fluid flow circuit 12 are connected by flexible tubing. The other components may include a plurality of fluid containers F1-F7. In the context of the present disclosure these containers include an anticoagulant container F1, a saline container F2, an in-process container F3, a return container F4, a plasma collection container F5, a platelet collection container F6, and an (optional) additive container F7. The illustrated flow circuit 12 further includes one or more blood source access devices (e.g., a connector for accessing blood within a fluid container or a phlebotomy needle), a spinning membrane separator 26 and a centrifugal separation chamber 36.

The flow control cassette provides a centralized, programmable, integrated platform for all the pumping and many of the valving functions required for a given blood processing procedure. In one embodiment, the cassette is similarly configured to the cassette of U.S. Pat. No. 5,868,696, but is adapted to include additional components (e.g., more tubing loops) and functionality.

In use, the cassette is mounted to the cassette station 54 of the blood separation device 10 so as to align each sensor station with an associated pressure sensor A1-A4 of the cassette station 54 and its valve stations with an associated valve V1-V9. Each valve station may define one or more ports that allow fluid communication between the valve station and another interior cavity of the cassette (e.g., a flow path). As described above, each valve V1-V9 is movable under command of the controller 18 to move between a plurality of positions (e.g., between a retracted or lowered position and an actuated or raised position) to selectively contact the valve stations of the cassette. In the actuated position, a valve V1-V9 engages the associated valve station to close one or more of its ports to prevent fluid flow therethrough. In the retracted position, a valve V1-V9 is disengaged from the associated valve station (or less forcefully contacts the associated valve station than when in the actuated position) to open one or more ports associated with the valve station, thereby allowing fluid flow therethrough.

A plurality of tubing loops extend from the side surface of the cassette to interact with pumps P1-P6 of the blood separation device 10. The different pumps P1-P6 may interact with the tubing loops of the cassette to perform different tasks during a separation procedure (as will be described in greater detail), but in the context of the present disclosure, a different one of the pumps P1-P6 may be configured to serve as an anticoagulant pump P1, a source pump P2, a centrifuge pump P3, an outlet pump P4, a recirculation pump P5, and a plasma pump P6.

Additional tubing extends from the side surface of the cassette to connect to the other components of the fluid flow circuit 12, such as the various fluid containers F1-F7, the spinning membrane separator 26, and the centrifugal separation chamber 36. The tubing connected to the centrifugal separator chamber 36 (which includes one inlet tube and two outlet tubes) may be aggregated into an umbilicus.

Various additional components may be incorporated into the tubing leading out of the cassette or into one of the cavities of the cassette. For example, a manual clamp 56 may be associated with a line or lines leading to the blood source/donor, a return line filter 58 (e.g., a microaggregate filter) may be associated with a line leading to a fluid recipient, and/or an air trap 62 may be positioned on a line upstream of the centrifugal separation chamber 36.

### III. Exemplary Platelet Separation And Collection Procedure

An exemplary platelet collection procedure for producing a platelet product having a final target platelet concentration according to the present disclosure will now be described.

Prior to processing, an operator selects the desired protocol (e.g., using an operator interface station, if provided), which informs the controller 18 of the manner in which it is to control the other components of the blood separation device 10 during the procedure. In the context of a platelet collection procedure, this includes whether plasma is also being collected (rather than being returned to the source), along with a first target concentration and a second target concentration (which is lower than the first target concentration). Rather than the operator selecting the first and second target concentrations, it is within the scope of the present disclosure for the first and second target concentrations to be selected by the controller 18. This may include the controller 18 providing the operator with suggested first and second target concentrations and the operator deciding whether to accept the suggested target concentrations. In any event, the nature and significance of the first target concentration and the second target concentration will be explained in greater detail below.

If the blood source is a donor, the operator may proceed to enter various parameters, such as the donor sex/height/weight. In one embodiment, the operator also enters the target yield or concentration for the various blood components (which may also include entering a characteristic of the blood, such as a platelet pre-count) or some other collection control factor (e.g., the amount of whole blood to be processed).

If there are any fluid containers (e.g., a platelet additive solution container) that are not integrally formed with the fluid flow circuit 12, they may be connected to the fluid flow circuit 12 (e.g., by piercing a septum of a tube of the fluid flow circuit 12 or via a luer connector), with the fluid flow circuit 12 then being mounted to the blood separation device 10 (including the fluid containers F1-F7 being hung from the weight scales W1-W6, as appropriate). An integrity check of the fluid flow circuit 12 may be executed by the controller 18 to ensure that the various components are properly connected and functioning. Following a successful integrity check, the blood source is connected to the fluid flow circuit 12 (e.g., by connecting to a container of previously collected whole blood or by phlebotomizing a donor), and the fluid flow circuit 12 may be primed (e.g., by saline pumped from a saline container F2 by operation of one or more of the pumps P1-P6 of the blood separation device 10).

After the fluid flow circuit 12 has been primed, blood separation may begin.

### A. Draw Phase

In a first phase (FIG. 3), blood is drawn into the fluid flow circuit 12 from a blood source. If the blood source is a donor, then blood may be drawn into the fluid flow circuit 12 through a single needle that is connected to the cassette by line L140. Line L140 may include a manual clamp 56 that may initially be in a closed position to prevent fluid flow through line L140. When processing is to begin, an operator may move the manual clamp 56 from its closed position to an open position to allow fluid flow through line L140.

The blood is drawn into line L140 by the source pump P2 of the blood separation device 10. Anticoagulant from the anticoagulant container F1 may be drawn through line L141 under action of the anticoagulant pump P1 and added to the blood at a junction of lines L140 and L141.

In the illustrated embodiment, valve V10 is open to allow blood to flow through lines L140 and L142 and a cassette sensor station associated with pressure sensor A1, while valve V11 is closed to prevent fluid flow through line L143. If the blood source is a living body (e.g., a donor), the pressure sensor A1 may communicate with the controller 18 to monitor the pressure within the vein of the blood source.

The cassette includes two valve stations downstream of the source pump P2 and line L144, with valve V2 being closed to prevent flow through line L145 and valve V1 being open to allow flow through line L146. The blood flows through line L146 to a junction, where a portion of the blood is directed through line L147 and a cassette sensor station associated with pressure sensor A3 to the in-process container F3 and the remainder is directed through line L148 toward the centrifuge pump P3, which controls the amount of blood that is directed to the centrifugal separation chamber 36 instead of the in-process container F3. In particular, the flow rate of the source pump P2 is greater than the flow rate of the centrifuge pump P3, with the difference therebetween being equal to the flow rate of blood into the in-process container F3. The flow rates may be selected such that the in-process container F3 is partially or entirely filled with blood at the end of the draw phase.

The blood pumped through line L148 by the centrifuge pump P3 passes through line L149, an air trap 62, and a cassette sensor station associated with pressure sensor A2 (which works in combination with the controller 18 of the blood separation device 10 to monitor the pressure in the centrifugal separation chamber 36) before reaching the centrifugal separation chamber 36 of the fluid flow circuit 12. The centrifugal separator 16 of the blood separation device 10 manipulates the centrifugal separation chamber 36 to separate the blood in the centrifugal separation chamber 36 into platelet-rich plasma and packed red blood cells. In one embodiment, the centrifugal separation chamber 36 is rotated nominally at 4,500 rpm, but the particular rotational speed may vary depending on the flow rates of fluids into and out of the centrifugal separation chamber 36.

The packed red blood cells exit the centrifugal separation chamber 36 via line L150 and flow through line L151 into the return container F4. White blood cells may be retained within the centrifugal separation chamber 36 or may exit with the red blood cells.

Platelet-rich plasma is drawn out of the centrifugal separation chamber 36 via line L152 by the combined operation of the recirculation and outlet pumps P5 and P4 of the blood separation device 10. The platelet-rich plasma travels through line L152 until it reaches a junction, which splits into lines L153 and L154. The recirculation pump P5 is associated with line L153 and redirects a portion of the platelet-rich plasma to a junction, where it mixes with blood in line L148 that is being conveyed into the centrifugal separation chamber 36 by the centrifuge pump P3. Recirculating a portion of the platelet-rich plasma into the centrifugal separation chamber 36 with inflowing blood decreases the hematocrit of the blood entering the centrifugal separation chamber 36, which may improve separation efficiency. By such an arrangement, the flow rate of the fluid entering the centrifugal separation chamber 36 is equal to the sum of the flow rates of the centrifuge pump P3 and the recirculation pump P5.

As the platelet-rich plasma drawn out of the centrifugal separation chamber 36 into line L153 by the recirculation pump P5 is immediately added back into the centrifugal separation chamber 36, the bulk or net platelet-rich plasma flow rate out of the centrifugal separation chamber 36 is equal to the flow rate of the outlet pump P4. Before reaching the spinning membrane separator 26, the portion of the platelet-rich plasma conveyed through line L154 by the outlet pump P4 passes through the spinner inlet sensor M1 and a cassette sensor station associated with pressure sensor A4. The spinner inlet sensor M1 may detect the concentration of platelets in the platelet-rich plasma entering the spinning membrane separator 26, while the pressure sensor A4 may monitor the pressure of the spinning membrane separator 26.

Line L154 has a junction, where it joins with line L155. Valve V6 is closed to prevent fluid flow through line L155, thereby directing the separated platelet-rich plasma to the spinning membrane separator 26. The valve V6 may be selectively opened to divert all or a portion of the platelet-rich plasma through line L155 and to the return container F4, if necessary. An example would be at the start of a procedure when separation is initializing and platelets are not yet exiting the centrifugal separation chamber 36, in which case the fluid conveyed through line L154 by the outlet pump P4 could be diverted to the return container F4.

The spinning membrane separator drive unit 14 of the blood separation device 10 manipulates the spinning membrane separator 26 to separate the platelet-rich plasma into platelet-poor plasma and platelet concentrate. Plasma is pumped out of the spinning membrane separator 26 via line L156 by the plasma pump P6 of the blood separation device 10. Valves V9, V8, V6, and V5 are closed to prevent flow through lines L157, L158, L155, and L159 (respectively), thereby directing the separated plasma along lines L156 and L160, through valve V4, and into the return container F4 (with the separated red blood cells). On the way to the return container F4, the plasma passes through spinner outlet sensor M2, which may cooperate with the controller 18 to determine one or more characteristics of the plasma, such as the amount of cellular blood components in the plasma and/or whether the plasma is hemolytic and/or lipemic.

The platelet concentrate is conveyed out of the spinning membrane separator 26 via line L161. There is no pump associated with line L161, so instead the flow rate at which the platelets exit the spinning membrane separator 26 is equal to the difference between the flow rates of the outlet pump P4 and plasma pump P6. Valve V8 is closed to prevent fluid flow through the line L158, thereby directing the flow of platelets along lines L161 and L162, through valve V7, and into the platelet collection container F6. Valve V8 may be selectively opened to allow fluid flow through line L158 and to a junction, where it joins the plasma flowing through line L156 to the return container F4, if necessary.

Notably, the controller 18 operates the other components of the blood separation device 10 to attempt to produce platelet concentrate having a target concentration. This is in contrast to the above-described conventional approach in which a blood separation system will simply collect all available platelets, regardless of the concentration of the resulting platelet concentrate. The concentration of the platelet concentrate is calculated according to the following equation:

PC concentration=(PRP concentration*PRP inlet rate)/(PRP inlet rate−PPP outlet rate), in which PC concentration is the concentration of the platelet concentrate, PRP concentration is the concentration of the platelet-rich plasma exiting the centrifugal separation chamber 36, PRP inlet rate is the rate at which the platelet-rich plasma is being pumped into the spinning membrane separator 26, and PPP outlet rate is the rate at which the platelet-poor plasma is pumped out of the spinning membrane separator 26. Thus, using this equation, the controller 18 may attempt to control the operation of the other components of the blood separation device 10 to produce platelet concentrate having a target concentration (which will be referred to herein as the “first target concentration”).

While the controller 18 will attempt to achieve the first target concentration, it may be the case that inaccuracies in the operation of the pump system may produce platelet concentrate having a different platelet concentration (referred to herein as the “actual platelet concentration” of the platelet concentrate). For example, a first target concentration may be 2000e3 platelets/μL. In this example, the blood of the blood source has a concentration of 220e3 platelets/μL and 5000 mL of blood is processed. The platelet concentration is multiplied by the volume of blood that is processed and by the known platelet separation efficiency of the blood separation device. The separation efficiency will vary from device to device, but will be treated as 80% in this example, which results in a total of 8.8e11 platelets being collected (220e3 platelets/μL×5000 mL of blood×80% efficiency).

In this example, 400 mL of platelet concentrate is collected by the end of separation, such that the actual platelet concentration of the collected platelets is 2200e3 platelets/μL (8.8e11 platelets/400 mL), rather than the target concentration of 2000e3 platelets/μL. As will be explained in greater detail, this difference between the actual platelet concentration and the first target concentration (due to the pump inaccuracy) will not prevent a final target concentration for the platelet product from being achieved.

In any event, the draw phase may continue until the amount of blood drawn from the blood source reaches a target amount or the in-process container F3 is filled to a particular level (as determined by a weight scale from which the in-process container F3 is hung during the procedure) or until some other condition is satisfied.

### B. Return Phase—Platelet Collection Only

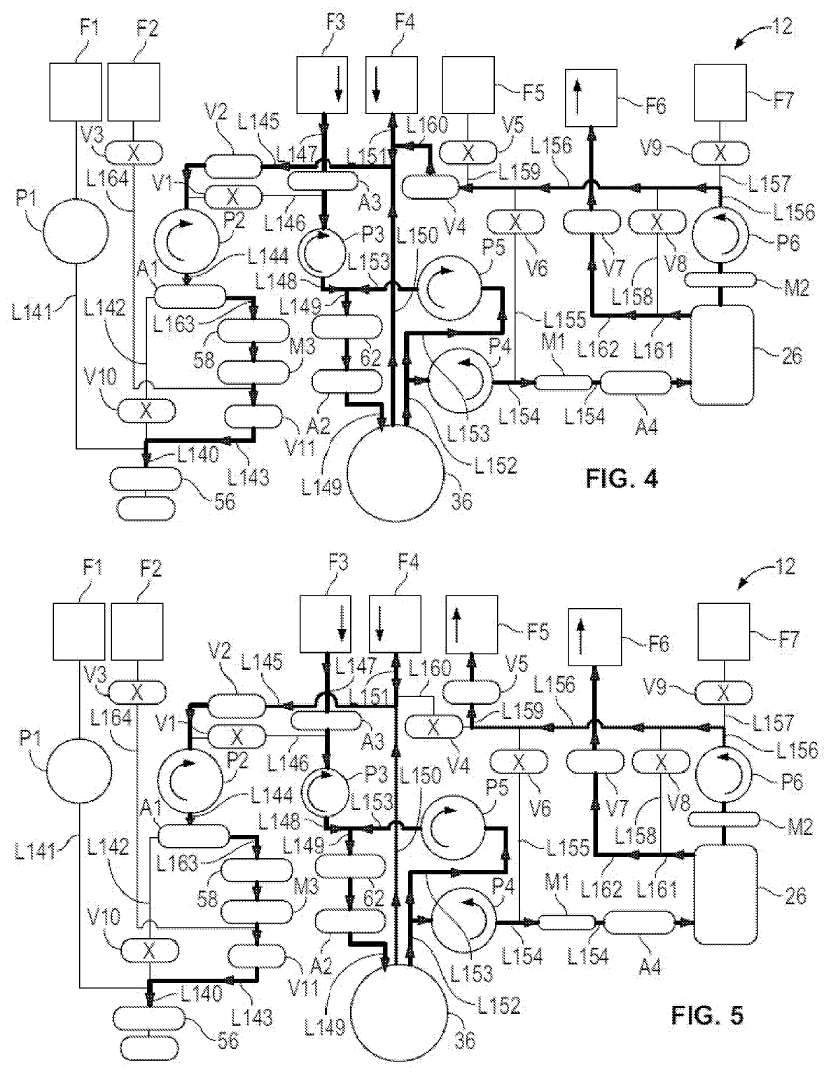

When the draw phase ends, the system transitions to one of two return phases (FIGS. 4 and 5), depending on whether only platelets are being collected (FIG. 4) or if both platelets and plasma are being collected (FIG. 5). At least a portion of one of the separated blood components is conveyed to a recipient (which may be the blood source), while the collection of either platelets (FIG. 4) or both platelets and plasma (FIG. 5) proceeds.

During the return phase of a procedure in which only platelets are being collected (FIG. 4), the anticoagulant pump P1 will stop drawing anticoagulant from the anticoagulant container F1. Valve V10 will close to prevent fluid flow through line L142 and valve V11 is opened to allow fluid flow through line L143. Valve V2 also opens to allow flow through line L145, while valve V1 is closed to prevent flow through line L146.

With the valves so situated, the source pump P2 will reverse direction to allow the contents of the return container F4 to be conveyed to a recipient via the same needle used to draw blood into the fluid flow circuit 12. The return fluid (red blood cells and plasma) is pumped through lines L151 and L145, valve V2, line L144, the cassette sensor station associated with pressure sensor A1, and into line L163. The return fluid travels along line L163, through a return line filter 58 and air detector M3 until it reaches a junction, which joins lines L163 and L164 (which leads to the saline container F2). Valve V3 is closed, thereby preventing fluid flow through line L164 and directing the return fluid further along line L163, through valve V11, and along lines L143 and L140 to the recipient.

While fluid is being conveyed to the recipient, the blood in the in-process container F3 acts as the blood supply for the centrifugal separator 16. When the system transitions to this return phase, operation of the centrifuge pump P3 remains unchanged and separation continues in the same manner as described for the draw phase (i.e., with blood being separated in the centrifugal separation chamber 36 into red blood cells and platelet-rich plasma, the red blood cells flowing out of the centrifugal separation chamber 36 to the return container F4, a portion of the platelet-rich plasma flowing to and being separated in the spinning membrane separator 26, and the separated platelets being collected while plasma is directed to the return container F4) until the in-process container F3 is emptied. Therefore, the system components downstream from the centrifuge pump P3 are “blinded” as to whether the system is in the draw phase or this return phase. It will be appreciated that a method as described herein is preferable to a batch process (by which blood is only separated during a draw phase and not during a return phase) because separation and collection may be continuous, thereby decreasing the time required to complete the procedure. Additionally, by continuously processing blood in the centrifugal separation chamber 36, the interface between the separated red blood cells and platelet-rich plasma is maintained, whereas the location of the interface is lost during the return phase of a batch procedure, which requires the interface to be reestablished during every draw/separation phase and further increases the duration of the procedure.

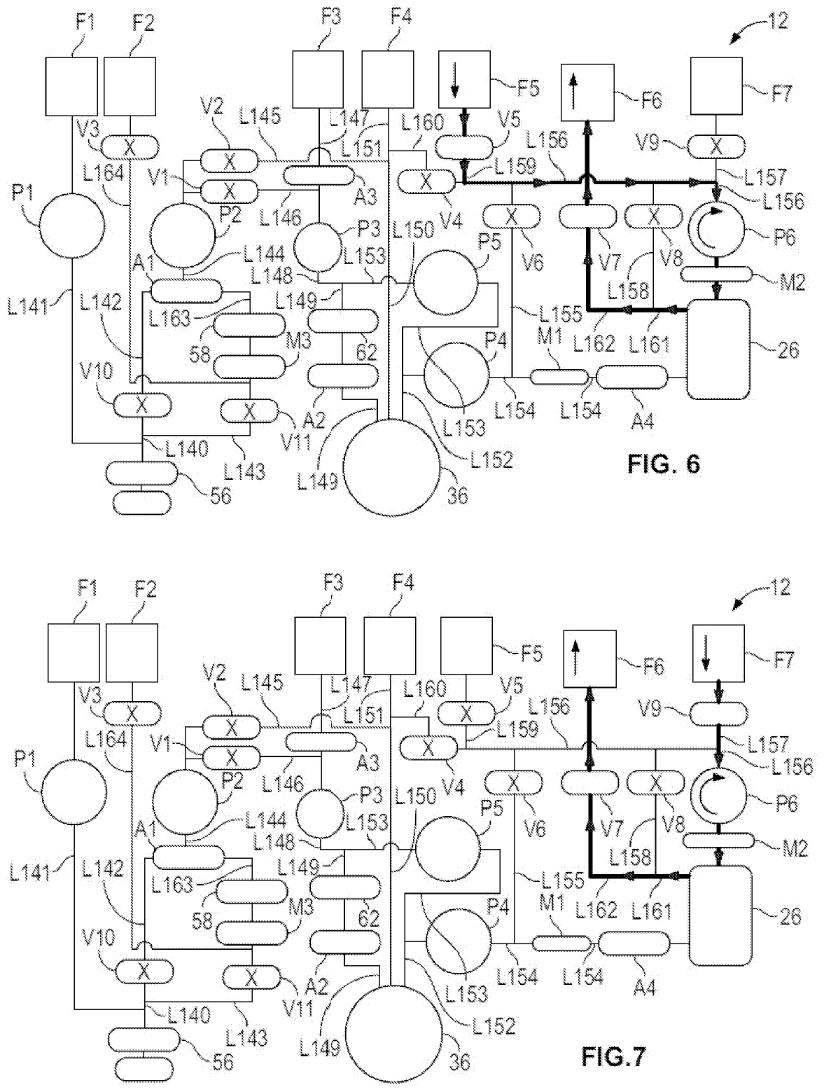

It will be seen that the separated plasma and red blood cells are conveyed to the return container F4 at the same time that the contents of the return container F4 are being conveyed to the recipient. The rate at which the source pump P2 operates may be greater than the rate at which plasma and red blood cells are conveyed into the return container F4 to allow the return container F4 to empty during this return phase, even as separation continues. Once the in-process and/or return container F3, F4 is empty, the system may transition back to the draw phase (FIG. 3) or to a plasma addition phase (FIG. 6) or an additive addition phase (FIG. 7).

### C. Return Phase—Platelet And Plasma Collection

The return phase when collecting platelets and plasma (FIG. 5) is similar to the return phase when only platelets are being collected (FIG. 4). The principal difference is that, when collecting platelets and plasma, valve V5 is open to allow flow through line L159, while valve V4 is closed to prevent flow through line L160, thereby directing the separated plasma exiting the spinning membrane separator 26 through the line L159 associated with open valve V5 to the plasma collection container F5 instead of to the return container F4. The valves may be so situated for the entire return phase or the conditions of valves V4 and V5 may be reversed for a portion of the phase (thereby directing separated plasma to the return container F4 instead of the plasma collection container F5), depending on the amount of plasma to be collected.

Once the in-process and/or return container F3, F4 is empty, the system may transition back to the draw phase (FIG. 3) or to a plasma addition phase (FIG. 6) or additive addition phase (FIG. 7).

### D. Volume Addition

When the targeted amount of platelets or the targeted amounts of platelets and plasma have been collected, the blood source/recipient may be disconnected from the fluid flow circuit 12 and the system may transition to a phase in which collected plasma (FIG. 6) or an additive solution (FIG. 7) is added to the collected platelets. It should be understood that the phases shown in FIGS. 6 and 7 are not exclusive, but that they may both be executed during the same platelet collection procedure (e.g., with amounts of both plasma and additive solution being added to the collected platelets).

In the case of dilution via plasma, this phase may be carried out with valves V5 and V7 in an open condition to allow flow through lines L159 and L156, while valves V9, V8, V6, and V4 are in a closed condition to prevent flow through lines L157, L158, L155, and L160 (FIG. 6). With the valves so situated, the plasma pump P6 may operate to draw the collected plasma from the plasma collection container F5 via line L159, through line L156, spinner outlet sensor M2, the spinning membrane separator 26, lines L161 and L162, open valve V7, and into the platelet collection container F6.

On the other hand, in the case of dilution using additive solution, this phase may be carried out with valves V9 and V7 in an open condition to allow flow through lines L157 and L162, while valves V8, V6, V5, and V4 are in a closed condition to prevent flow through lines L158, L155, L159, and L160 (FIG. 7). With the valves so situated, the plasma pump P6 may operate to draw the platelet additive solution from the additive container bag F7 via line L157, through open valve V9, line L156, spinner outlet sensor M2, the spinning membrane separator 26, lines L161 and L162, open valve V7, and into the platelet collection container F6.

This phase continues until the fluid or platelet product in the platelet collection container F6 has a second or final target concentration, which coincides with the platelet product also having a target volume (as determined by a weight scale associated with the platelet collection container F6).

Continuing the above-described example (in which collected platelet concentrate has an actual platelet concentration of 2200e3 platelets/μL instead of the first target concentration of 200e3 platelets/μL), the controller 18 will determine the volume of plasma and/or additive solution to be added to the collected platelets during this phase to achieve the second or final target concentration. In this example, the second or final target concentration for the platelet product is 1500e3 platelets/μL. As noted above, the total number of platelets collected in this example is 8.8e11 platelets, such that the final volume of the platelet product at the end of the procedure must be 586.7 mL (8.8e11 platelets/1500e3 platelets/μL). At the end of blood separation and platelet collection, the collected platelet concentrate (in this example) has a volume of 400 mL, such that 186.7 mL of plasma and/or additive solution must be added during this phase (586.7 mL-400 mL). Upon this target volume for the platelet product being achieved, the final or second target platelet concentration will also be achieved. Hence, it will be seen that the pump inaccuracy when collecting platelets (which results in platelet concentrate having an actual platelet concentration that differs from the first target concentration) does not prevent the final or second target concentration from being achieved, as the volume addition phase will dictate the final concentration by bringing the platelet product volume up to the value needed to lower the concentration from the actual platelet concentration at the end of collection (2200e3 platelets/μL, in this example) to the second or final target concentration (1500e3 platelets/μL, in this example) for the platelet product at the end of the procedure.

### IV. Aspects

Aspect 1. A blood separation device comprising: a pump system; a valve system; a separation system; and a controller configured and/or programmed to control the operation of the pump system, the valve system, and the separation system to execute a platelet collection procedure comprising: selecting a first target concentration and a second target concentration, separating blood into red blood cells and platelet-rich plasma, separating the platelet-rich plasma into platelet-poor plasma and platelet concentrate, collecting at least a portion of the platelet concentrate in a container as a platelet product having an actual platelet concentration, with the pump system being controlled so as to attempt to collect platelet concentrate having the first target concentration, and after the separation of blood has been completed, pumping at least a portion of the platelet-poor plasma and/or an additive solution into the container to decrease the concentration of the platelet product from the actual platelet concentration to the second target concentration.

Aspect 2. The blood separation device of Aspect 1, wherein the separation system includes a centrifugal separator.

Aspect 3. The blood separation device of any one of the preceding Aspects, wherein the separation system includes a spinning membrane separator drive unit.

Aspect 4. The blood separation device of Aspect 1, wherein the separation system includes a centrifugal separator configured to be controlled by the controller to separate the blood into red blood cells and platelet-rich plasma, and a spinning membrane separator drive unit configured to be controlled by the controller to separate the platelet-rich plasma into platelet-poor plasma and platelet concentrate.

Aspect 5. The blood separation device of any one of the preceding Aspects, wherein at least a portion of the platelet-rich plasma is separated simultaneously with a portion of the blood being separated.

Aspect 6. The blood separation device of any one of the preceding Aspects, wherein PC concentration=(PRP concentration*PRP inlet rate)/(PRP inlet rate−PPP outlet rate), in which PC concentration is a concentration of the platelet concentrate, PRP concentration is a concentration of the platelet-rich plasma, PRP inlet rate is a rate at which the platelet-rich plasma is being pumped prior to separation into platelet-poor plasma and platelet concentrate, and PPP outlet rate is a rate at which the platelet-poor plasma is pumped following separation of the platelet-rich plasma.

Aspect 7. The blood separation device of any one of the preceding Aspects, wherein the controller is configured and/or programmed to execute the platelet collection procedure such that the platelet product reaches a target volume upon the platelet product reaching the second target concentration.

Aspect 8. A blood separation device comprising: a pump system; a valve system; a centrifugal separator; a spinning membrane separator drive unit; and a controller configured and/or programmed to control the operation of the pump system, the valve system, the centrifugal separator, and the spinning membrane separator drive unit to execute a platelet collection procedure comprising: selecting a first target concentration and a second target concentration, pumping blood from a blood source into the centrifugal separator, separating the blood in the centrifugal separator into red blood cells and platelet-rich plasma, pumping at least a portion of the platelet-rich plasma from the centrifugal separator into the spinning membrane separator drive unit, separating the platelet-rich plasma in the spinning membrane separator drive unit into platelet-poor plasma and platelet concentrate, collecting at least a portion of the platelet-poor plasma in a first container as collected plasma and at least a portion of the platelet concentrate in a second container as a platelet product having an actual platelet concentration, with the pump system being controlled so as to attempt to collect platelet concentrate having the first target concentration, and after the separation of blood has been completed, pumping at least a portion of the collected plasma and/or an additive solution into the second container to decrease the concentration of the platelet product from the actual platelet concentration to the second target concentration.

Aspect 9. The blood separation device of Aspect 8, wherein PC concentration=(PRP concentration*PRP inlet rate)/(PRP inlet rate−PPP outlet rate), in which PC concentration is a concentration of the platelet concentrate, PRP concentration is a concentration of the platelet-rich plasma, PRP inlet rate is a rate at which the platelet-rich plasma is pumped into the spinning membrane separator drive unit, and PPP outlet rate is a rate at which the platelet-poor plasma is pumped out of the spinning membrane separator drive unit.

Aspect 10. The blood separation device of any one of Aspects 8-9, wherein the controller is configured and/or programmed to execute the platelet collection procedure such that the platelet product reaches a target volume upon the platelet product reaching the second target concentration.

Aspect 11. The blood separation device of any one of Aspects 8-10, further comprising an inlet sensor configured to optically detect a characteristic of the platelet-rich plasma to be separated in the spinning membrane separator drive unit.

Aspect 12. The blood separation device of Aspect 11, wherein the inlet sensor is configured to optically detect the concentration of platelets in the platelet-rich plasma to be separated in the spinning membrane separator drive unit.

Aspect 13. The blood separation device of any one of Aspects 8-12, wherein the controller is configured and/or programmed to simultaneously control the centrifugal separator to separate the blood and control the spinning membrane separator drive unit to separate the platelet-rich plasma.

Aspect 14. A blood separation method, comprising: selecting a first target concentration and a second target concentration; separating blood into red blood cells and platelet-rich plasma; separating the platelet-rich plasma into platelet-poor plasma and platelet concentrate; collecting at least a portion of the platelet concentrate in a container as a platelet product having an actual platelet concentration, attempting to collect platelet concentrate having the first target; and after the separation of blood has been completed, pumping at least a portion of the platelet-poor plasma and/or an additive solution into the container to decrease the concentration of the platelet product from the actual platelet concentration to the second target concentration.

Aspect 15. The blood separation method of Aspect 14, wherein said separating blood into red blood cells and platelet-rich plasma includes separating the blood using a centrifugal separator.

Aspect 16. The blood separation method of Aspect 14, wherein said separating platelet-rich plasma into platelet-poor plasma and platelet concentrate includes separating the platelet-rich plasma using a spinning membrane separator drive unit.

Aspect 17. The blood separation method of Aspect 14, wherein said separating blood into red blood cells and platelet-rich plasma includes separating the blood using a centrifugal separator, and said separating platelet-rich plasma into platelet-poor plasma and platelet concentrate includes separating the platelet-rich plasma using a spinning membrane separator drive unit.

Aspect 18. The blood separation method of any one of Aspects 14-17, wherein at least a portion of the platelet-rich plasma is separated simultaneously with a portion of the blood being separated.

Aspect 19. The blood separation method of any one of Aspects 14-18, wherein PC concentration=(PRP concentration*PRP inlet rate)/(PRP inlet rate−PPP outlet rate), in which PC concentration is a concentration of the platelet concentrate, PRP concentration is a concentration of the platelet-rich plasma, PRP inlet rate is a rate at which the platelet-rich plasma is being pumped prior to separation into platelet-poor plasma and platelet concentrate, and PPP outlet rate is a rate at which the platelet-poor plasma is pumped following separation of the platelet-rich plasma.

Aspect 20. The blood separation method of any one of Aspects 14-19, wherein the platelet product reaches a target volume upon the platelet product reaching the second target concentration.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A blood separation device comprising:
a pump system;
a valve system;
a separation system; and
a controller configured and/or programmed to control the operation of the pump system, the valve system, and the separation system to execute a platelet collection procedure comprising:
selecting a first target concentration and a second target concentration, separating blood into red blood cells and platelet-rich plasma,
separating the platelet-rich plasma into platelet-poor plasma and platelet concentrate,
collecting at least a portion of the platelet concentrate in a container as a platelet product having an actual platelet concentration, with the pump system being controlled so as to attempt to collect platelet concentrate having the first target concentration, and
after the separation of blood has been completed, pumping at least a portion of the platelet-poor plasma and/or an additive solution into the container to decrease the concentration of the platelet product from the actual platelet concentration to the second target concentration.

2. The blood separation device of claim 1, wherein the separation system includes a centrifugal separator.

3. The blood separation device of claim 1, wherein the separation system includes a spinning membrane separator drive unit.

4. The blood separation device of claim 1, wherein the separation system includes
a centrifugal separator configured to be controlled by the controller to separate the blood into red blood cells and platelet-rich plasma, and
a spinning membrane separator drive unit configured to be controlled by the controller to separate the platelet-rich plasma into platelet-poor plasma and platelet concentrate.

5. The blood separation device of claim 1, wherein at least a portion of the platelet-rich plasma is separated simultaneously with a portion of the blood being separated.

6. The blood separation device of claim 1, wherein
PC concentration=(PRP concentration*PRP inlet rate)/(PRP inlet rate−PPP outlet rate), in which
PC concentration is a concentration of the platelet concentrate,
PRP concentration is a concentration of the platelet-rich plasma,
PRP inlet rate is a rate at which the platelet-rich plasma is being pumped prior to separation into platelet-poor plasma and platelet concentrate, and
PPP outlet rate is a rate at which the platelet-poor plasma is pumped following separation of the platelet-rich plasma.

7. The blood separation device of claim 1, wherein the controller is configured and/or programmed to execute the platelet collection procedure such that the platelet product reaches a target volume upon the platelet product reaching the second target concentration.

8. A blood separation device comprising:
a pump system;
a valve system;
a centrifugal separator;
a spinning membrane separator drive unit; and
a controller configured and/or programmed to control the operation of the pump system, the valve system, the centrifugal separator, and the spinning membrane separator drive unit to execute a platelet collection procedure comprising:
selecting a first target concentration and a second target concentration,
pumping blood from a blood source into the centrifugal separator,
separating the blood in the centrifugal separator into red blood cells and platelet-rich plasma,
pumping at least a portion of the platelet-rich plasma from the centrifugal separator into the spinning membrane separator drive unit,
separating the platelet-rich plasma in the spinning membrane separator drive unit into platelet-poor plasma and platelet concentrate,
collecting at least a portion of the platelet-poor plasma in a first container as collected plasma and at least a portion of the platelet concentrate in a second container as a platelet product having an actual platelet concentration, with the pump system being controlled so as to attempt to collect platelet concentrate having the first target concentration, and
after the separation of blood has been completed, pumping at least a portion of the collected plasma and/or an additive solution into the second container to decrease the concentration of the platelet product from the actual platelet concentration to the second target concentration.

9. The blood separation device of claim 8, wherein
PC concentration=(PRP concentration*PRP inlet rate)/(PRP inlet rate−PPP outlet rate), in which
PC concentration is a concentration of the platelet concentrate,
PRP concentration is a concentration of the platelet-rich plasma,
PRP inlet rate is a rate at which the platelet-rich plasma is pumped into the spinning membrane separator drive unit, and
PPP outlet rate is a rate at which the platelet-poor plasma is pumped out of the spinning membrane separator drive unit.

10. The blood separation device of claim 8, wherein the controller is configured and/or programmed to execute the platelet collection procedure such that the platelet product reaches a target volume upon the platelet product reaching the second target concentration.

11. The blood separation device of claim 8, further comprising an inlet sensor configured to optically detect a characteristic of the platelet-rich plasma to be separated in the spinning membrane separator drive unit.

12. The blood separation device of claim 11, wherein the inlet sensor is configured to optically detect the concentration of platelets in the platelet-rich plasma to be separated in the spinning membrane separator drive unit.

13. The blood separation device of claim 8, wherein the controller is configured and/or programmed to simultaneously control the centrifugal separator to separate the blood and control the spinning membrane separator drive unit to separate the platelet-rich plasma.

14. A blood separation method, comprising:
selecting a first target concentration and a second target concentration;
separating blood into red blood cells and platelet-rich plasma;
separating the platelet-rich plasma into platelet-poor plasma and platelet concentrate;
collecting at least a portion of the platelet concentrate in a container as a platelet product having an actual platelet concentration, attempting to collect platelet concentrate having the first target concentration; and
after the separation of blood has been completed, pumping at least a portion of the platelet-poor plasma and/or an additive solution into the container to decrease the concentration of the platelet product from the actual platelet concentration to a second target concentration.

15. The blood separation method of claim 14, wherein said separating blood into red blood cells and platelet-rich plasma includes separating the blood using a centrifugal separator.

16. The blood separation method of claim 14, wherein said separating platelet-rich plasma into platelet-poor plasma and platelet concentrate includes separating the platelet-rich plasma using a spinning membrane separator drive unit.

17. The blood separation method of claim 14, wherein
said separating blood into red blood cells and platelet-rich plasma includes separating the blood using a centrifugal separator, and
said separating platelet-rich plasma into platelet-poor plasma and platelet concentrate includes separating the platelet-rich plasma using a spinning membrane separator drive unit.

18. The blood separation method of claim 14, wherein at least a portion of the platelet-rich plasma is separated simultaneously with a portion of the blood being separated.

19. The blood separation method of claim 14, wherein
PC concentration=(PRP concentration*PRP inlet rate)/(PRP inlet rate−PPP outlet rate), in which
PC concentration is a concentration of the platelet concentrate,
PRP concentration is a concentration of the platelet-rich plasma,
PRP inlet rate is a rate at which the platelet-rich plasma is being pumped prior to separation into platelet-poor plasma and platelet concentrate, and
PPP outlet rate is a rate at which the platelet-poor plasma is pumped following separation of the platelet-rich plasma.

20. The blood separation method of claim 14, wherein the platelet product reaches a target volume upon the platelet product reaching the second target concentration.
